SEQUENCE LISTING (9 sequences)

SEQ ID NO: 1 - DNA, 27 bp, Red Sea Bream Iridovirus
  misc_feature (1)..(27): n=inosine
ggtncanggtctangagacnncccggg

SEQ ID NO: 2 - DNA, 18 bp, Homo sapiens
actccaccatagatcact

SEQ ID NO: 3 - DNA, 22 bp, homo sapiens
  misc_feature (1)..(22): n=inosine
ctanccatggcnttagtatgag

SEQ ID NO: 4 - DNA, 21 bp, homo sapiens
  misc_feature (1)..(21): n=inosine
agcaccctntcaggcagtacc

SEQ ID NO: 5 - DNA, 20 bp, Helianthus tuberosus
  misc_feature (1)..(20): n=inosine
tgggtcncgaaagnccttgt

SEQ ID NO: 6 - DNA, 25 bp, Drosophila melanogaster
  misc_feature (1)..(25): n=inosine
gctcatgntgcacgnnctncgagac

SEQ ID NO: 7 - DNA, 20 bp, Cyprinella galactura
  misc_feature (1)..(20): n=inosine
catagntcactcccctgtga

SEQ ID NO: 8 - DNA, 25 bp, Bovine viral diarrhea virus
  misc_feature (1)..(25): n=inosine
aaagngnctagccatgncnttagta

SEQ ID NO: 9 - DNA, 31 bp, Helianthus tuberosus
  misc_feature (1)..(31): n=inosine
gtacctgggtcncgaaagnccttgtggtact

(19) United States
(12) Patent Application Publication
(10) Pub. No.: US 2002/0164596 A1
(43) Pub. Date: Nov. 7, 2002

(54) Proccess for finding oligonuclestide sequences for nucleic acid amplification methods

(75) Inventor: Thomas Weimer, Gladenbach (DE)

Correspondence Address: FINNEGAN, HENDERSON, FARABOW, GARRETT &, DUNNER LLP, 1300 I STREET, NW, WASHINGTON, DC, 20005, US

(73) Assignee: AVENTIS BEHRING GMBH

(21) Appl. No.: 09/833,675

(22) Filed: Apr. 13, 2001

(30) Foreign Application Priority Data
Apr. 14, 2000 (DE) 100 18 901.6
May 19, 2000 (DE) 100 24 830.6

(51) Int. Cl. C12Q 1/70; C12Q 1/68; G06F 19/00

(52) U.S. Cl. 435/6; 435/5; 702/20

(57) ABSTRACT

A process for finding heterologous oligonucleotide sequences for a nucleic acid amplification method is described, in which process

a) mutually overlapping oligonucleotide sequences are generated by fragmenting conserved regions of the target nucleic acid to be amplified,

b) these sequence fragments are used for finding similar DNA segments in Genbank or other DNA databases and suitable heterologous oligonucleotide sequences which are derived from organisms of other species are thereby identified, and

c) the heterologous oligonucleotide sequences which have been found are employed as primers and/or probes for isolating the target nucleic acid using a nucleic acid amplification method.

The invention relates to a process for finding heterologous oligonucleotide sequences which are suitable for detecting a specific, predetermined and precisely known target nucleic acid including unknown variants and mutants of this target nucleic acid.

Nucleic acid amplification methods (NAT), such as PCR (polymerase chain reaction), NASBA (=nucleic acid sequence-based amplification), TMA (transcription-mediated amplification) and LCR (ligase chain reaction), inter alia, are efficient methods for accumulating large quantities of a specific DNA sequence in vitro and thereby making it available for analysis. Since any arbitrary DNA segment can be amplified, these methods, above all PCR, have been applied in many different ways and are also used, inter alia, for detecting viral contaminants which may be present in blood or blood plasma. The use of NAT to examine plasma products for the presence of viral nucleic acids, in order to increase the viral safety of these products, has therefore become established practice. This method can be used in virus diagnosis to detect viral genomes directly in patient blood before viral proteins, or antibodies against them, can be detected in the blood.

PCR is based on three reaction steps being repeated many times: the reaction mixture containing, as the template, double-stranded DNA having the sought-after sequence is denatured by heat and the two single strands are dissociated. On cooling, the primers, which have been added in excess, hybridize with the complementary base sequences on the template DNA. The primers used in this context are synthetic oligonucleotides which contain from 15 to 30 bases and which possess sequences which are complementary to the beginning and the end of the sought-after DNA segment. The sought-after DNA segment is therefore flanked by the two primers. In the third reaction step, the temperature is brought to the optimum for the heat-stable DNA polymerase. Starting from the primers, the polymerase synthesizes one copy per starting DNA, with the length of the DNA to be duplicated being determined by the distance between the primers. By repeating these process steps many times, the target DNA is amplified and made available for analysis.

Primer sequences which hybridize with in each case one of the DNA strands, at the two ends of the DNA segment to be amplified, are a prerequisite for performing a PCR in a target-orientated manner. For this reason, it is necessary to have precise knowledge of the nucleotide sequences at the beginning and end of the DNA segment to be duplicated. In order to prepare suitable primers, therefore, it has until now been regarded as being necessary to prepare a primer which is suitable for selective hybridization and whose adequate hybridization with the material under investigation is ensured by the primer having a species-specific, i.e. autologous nucleotide sequence.

In this connection, selective hybridization means that such a primer, which hybridizes selectively, hybridizes only, and exclusively, with the DNA segment to be detected, i.e. the target nucleic acid.

However, it has now turned out that the conventional PCR method is limited in its application range by being tied down to species-specific, autologous primers and to the autologous oligonucleotide probes which are likewise employed in this method, and is, on occasion, not suitable for identifying unknown variants of the DNA sequence to be detected.

The object therefore arose of improving the application range of nucleic acid amplification methods, in particular the PCR method, by making available, for detecting a target nucleic acid, a selection of primers which hybridize nonselectively with this target nucleic acid. This object is achieved by means of a process for obtaining heterologous primers, which hybridize nonselectively with the target nucleic acid, from organisms which are foreign with regard to the target nucleic acid, with these primers nevertheless being suitable for amplifying a target nucleic acid.

The invention therefore relates to a process for finding heterologous oligonucleotide sequences for a nucleic acid amplification method, in which

a) mutually overlapping sequence fragments, which preferably comprise from 30 to 50 bases (e.g. from 1 to 50, from 25 to 75, from 50 to 100, etc.), are generated by fragmenting conserved regions of the nucleic acid to be amplified,

b) these sequence fragments are used for finding similar DNA segments in Genbank or other DNA databases, e.g. EMBL, and heterologous, i.e. hybridizing oligonucleotide sequences which are derived from organisms of other species are thereby identified, and

c) the heterologous, hybridizing oligonucleotide sequences which have been found are employed as primers and/or probes for isolating the target nucleic acid using a nucleic acid amplification method.

This process can be particularly advantageously used for detecting viral sequence segments by generating mutually overlapping sequence fragments by fragmenting preferably conserved regions of the genome of a virus and identifying oligonucleotide sequences which hybridize with these fragments, and which are derived from organisms of other species, in a gene library. The sequence fragments should preferably possess from 30 to 50 bases.

The cleavage is effected on the basis of the observation that, while many homology search programs (such as FastN, Blast or Wordsearch, forming part of the Genetics Computer Group Inc. Wisconsin Package) are designed to find sequence similarities or homologies with respect to complete genes or relatively large DNA sequences, the task when searching for primers and probes which are suitable for nucleic acid technologies consists precisely in finding short sequences which possess a very high degree of similarity. It is also advisable to exclude the target virus sequences from the homology search from the outset in order to increase the prospects of successfully identifying as many heterologous sequences as possible.

The sequences which are found in a gene library in association with the above-described homology investigations exhibit different degrees of homology and sequence lengths which can differ from those of the fragmented oligonucleotide sequences employed. These heterologous oligonucleotide sequences have therefore to be subsequently checked carefully, once again, for their suitability for use as primers and probes. The total length of the homologous sequence, the number of consecutive nucleotides, the number of mismatches which are present, its G/C content and the calculated denaturing temperature, as a measure of the strength of the binding of the primer or the probe to the DNA, are important criteria for determining the suitability of the heterologous sequences for replacing the autologous primers and probes.

If the heterologous primers which have been obtained in this way still contain mismatches, the bases which are located at these points can then be replaced with a “universal base”, such as inosine, thereby making it possible to achieve complete hybridization of the nucleotide sequence of the heterologous primer with the target nucleic acid.

It is consequently possible to use this process to obtain heterologous primers from organisms of other species, with these primers being suitable for hybridization with the target nucleic acid in the same way as autologous primers, i.e. primers which are derived from the DNA sequences present in the organism which contains the target nucleic acid. The heterologous oligonucleotides which have been obtained in this way can also be used for preparing a probe which can be employed for a PCR. Such probes are frequently fluorescence-labeled and are based, for example, on a 5′-nuclease assay (Livak et al., 1995. Oligonucleotides with fluorescent dyes at opposite ends provide a quenched probe system useful for detecting a PCR product and nucleic acid hybridization. PCR Meth. Applic. 4:357-362) or on a particular secondary structure (Tyagi et al., Molecular beacons: Probes that Fluoresce upon Hybridization. Nature Biotechnology. March 1996, Vol. 14, pages 303-308).

However, it is also possible, for the process according to the invention, to employ a primer which is labeled with two fluorescent dyes (reporter and quencher), with this primer not hybridizing completely with the DNA sequence to be amplified at the 3′ end. This method is described in German patent application 197 55 642.6. In the amplification, for which it is possible to employ one or more thermostable DNA polymerases, at least one of which must have proof-reading properties, the unpaired bases of the labeled primer are then liberated together with the quencher dye attached thereto, resulting in an increase in the fluorescence at the wavelength of the reporter dye.

The heterologous oligonucleotides which are obtained by the process according to the invention, and which can be employed for a PCR, are consequently distinguished by the fact that, when used as primers or probes, they hybridize not only with the DNA sequence of the target nucleic acid but also with nucleic acids present in organisms of other species. Despite this, they are suitable for detecting or isolating the target nucleic acid in exactly the same way as autologous oligonucleotides, that is oligonucleotides which are derived from the same organism.

Employing the above-described approach, the conserved 5′-untranslated region of hepatitis C virus (HCV) was used for finding heterologous sequences. The following sequences represent a selection of the search results and were used for detecting HCV by means of PCR.

The SEQ IDs firstly show the derived primer sequences (SEQ IDs 1, 2, 3, 4, 6, 7, 8) or probe sequences (5, 9) and, below that, the homology of the respective HCV region with the heterologous sequence. In the sequences which were used, the nonhomologous bases were replaced with inosine (I).

```
SEQ ID No.1:
^{5′} GGT ICA IGG TCT AIG AGA CII CCC GGG^{3′}
AB007366 Red Sea Bream Iridovirus gene for DNA polymerase
  345 ..TCATGGTGCACGGTCTACGAGACCTCCCGGG... 315
                      ||| || |||||| ||||| ||||||
1184 AGCATGGGTTCAGGGTCTATGAGACGCCCCGGGCGT 1219
```

where “^{5′} GGT ICA IGG TCT AIG AGA CII CCC GGG ^{3′}” depicts the sequence of a primer. AB007366 is the accession number in GenBank under which the sequence of the Red Sea Bream iridovirus DNA polymerase gene is deposited. The sequence comparison shows the homology which exists between HCV (top) and the DNA polymerase gene (bottom), with a denoting an identical base.

```
SEQ ID No.2:
^{5′} ACT CCA CCA TAG ATC ACT ^{3′}
AB020864 Homo sapiens genomic DNA of 8p21.3-p22
      31 GGAGTGATCTATGGTGGAGT 12
            | ||||||||||||||||||
95982 GCAGTGATCTATGGTGGAGT 96001
```

```
SEQ ID No.3:
^{5′} CTA ICC ATG GCI TTA GTA TGA G ^{3′}
AC004616 Homo sapiens Xp22
      88 CTCATACTAACGCCATGGCTAG 67
            |||||||||| ||||||| |||
80839 CTCATACTAAAGCCATGGATAG 80860
```

```
SEQ ID No.4:
^{5′} AGC ACC CTI TCA GGC AGT ACC
Z97055 Human DNA sequence from PAC 388M5 on chromosome 22
    225 .GGTACTGCCTGATAGGGTGCTTGCGAGTGCC ... 315
              |||||||||||| |||||||| | |
50941 TGGTACTGCCTGAGAGGGTGCTGCTGCCTTTGGGA 50975
```

SEQ ID No.5:
^{5′} FAM-TGG GTC ICG AAA GIC CTT GT-TAMRA ^{3′}
AJ009757 Helianthus tuberosus sst-1 gene
274 CCACAAGGCCTTTCGCGACCCAAC 251
| |||||| |||||| |||||| ||||||| |||||| |||||| |
711 CTACAAGGACTTTCGGGACCCATC 734

```
SEQ ID No.6:
^{5′} GCT CAT GIT GCA CGI ICT ICG AGA C ^{3′}
AJ0J.0298 Drosophila melanogaster retrotransposon-like element
  335 GCTCATGATGCACGGTCTACGAGAC 311
          ||||||| |||||| || ||||||
4557 GCTCATGGTGCACGAGCTCCGAGAC 4581
```

```
SEQ ID No.7:
^{5′} CAT AGI TCA CTC CCC TGT GA 3′
AF111207 Cyprinella galactura NADH dehydrogenase subunit 2 (ND2)
gene
  60 .CAGTAGTTCCTCACAGGGGAGTGATCTATGG... 30
            | || |||||||||||||| |||||
228 CAATGCGTGGATCACAGGGGAGTGAACTATGACTA 262
```

SEQ ID No.8:
^{5′} AAA GIG ICT AGC CAT GIC ITT AGT A ^{3′}
BVDCG Bovine viral diarrhea virus complete genome.
60 ...AAAGCGTCTAGCCATGGCGTTACTATGATG 89
|||| | ||||||||| | |||||| ||
92 CGAAAAGAGGCTAGCCATGCCCTTAGTAGGACT 124

SEQ ID No.9:
^{5′} FAM-GTA CCT GGG TCI CGA AAG ICC TTG TGG TAC T-
TAMRA ^{3′}
AJ009757 Helianthus tuberosus sst-1 gene
274 CCACAAGGCCTTTCGCGACCCAAC 251
| |||||| |||||| |||||| |
711 CTACAAGGACTTTCGGGACCCATC 734

If the abovementioned primers and probes are used for a PCR, a nucleic acid amplification can then only take place in the presence of the hepatitis C virus nucleic acid since a prerequisite for the PCR is that at least one primer pair hybridizes with the nucleic acid to be amplified. However, in the present case, that is only possible in the presence of hepatitis C virus since the requisite primer pair for any other nucleic acid is not available. A nested PCR increases the specificity still further.

The following reaction mixtures were used to demonstrate the specificity and sensitivity of the above-described primers and probes with regard to detecting HCV.

### EXAMPLE 1

### HCV Nested PCR, TagMan Detection

RNA Extraction

In order to detect hepatitis C virus RNA, this RNA is firstly extracted using standard methods (e.g. Ishizawa M., Kobayashi Y., Miyamura T., Matsuma, S: Simple procedure of DNA isolation from human serum. Nucl. Acids Res. 1991; 19:5792). The amplification is set up as follows:

cDNA Synthesis

Ten μl of the extracted RNA are mixed with 4 μl of 5× First Strand Buffer (Life Technologies), 2 μl of primer 1 (50 pmol/μl; see SEQ ID No. 1 in the sequence listing), 1 μl of dNTPs (10 mM), 2 μl of dithiothreitol (100 mM), 0.75 μl of water and 0.25 μl of Superscript (50 units, Life Technologies), and the mixture is incubated at 42° C. for one hour. The enzyme is then inactivated at 95° C. for 5 minutes.

1st PCR

80 μl of a 1st PCR reaction mixture [61.7 μl of water, 8 μl of 10× PCR buffer (Perkin Elmer), 2 μl of primer 2 (50 pmol/μl; see SEQ ID No. 2 in the sequence listing), 4.8 μl of magnesium chloride (25 mM), 3 μl of dNTPs (2.5 mM), 0.5 μl of Taq DNA polymerase (2.5 units, Perkin Elmer)] are pipetted into the cDNA mixture and the whole is mixed and subjected to the following thermocycles:

1. Initial denaturation for 1 minute at 90° C.

2. 35 cycles of in each case 28 seconds at 94° C. (denaturation), 28 seconds at 50° C. (annealing) and 38 seconds at 60° C. (extension)

2nd PCR

Five μl of the 1st PCR mixture are mixed with 45 μl of a 2nd PCR reaction mixture [16.55 μl of water, 5 μl of 10× PCR buffer (Perkin Elmer), 3 μl of magnesium chloride (25 mM), 4 μl of dNTPs (2.5 mM), 8 μl of primer 3 (10 pmol/μl; see SEQ ID No. 3 in the sequence listing), 8 μl of primer 4 (10 pmol/μl; see SEQ ID No. 4 in the sequence listing), 0.25 μl of the TaqMan probe 5 (10 pmol/μl; see SEQ ID No. 5 in the sequence listing), 0.2 μl of Taq DNA polymerase (2.5 units, Perkin Elmer)] and the mixture is subjected to the following thermocycles:

1. Initial denaturation for 1 minute at 90° C.

2. 35 cycles of in each case 28 seconds at 94° C. (denaturation), and 1 minute at 56° C. (annealing and extension)

3. Cooling at 4° C. until evaluation.

Evaluation

The PCR reaction is evaluated in a fluorescence spectrometer. For this, the fluorescence is measured at the reporter wavelength (518 nm for FAM). A threshold value is calculated on the basis of the fluorescence of negative controls which do not contain any target sequence and unknowns are evaluated against this value.

### EXAMPLE 2

### HCV Nested PCR, Detection by means of Molecular Beacons

RNA Extraction

In order to detect hepatitis C virus RNA, this RNA is firstly extracted using standard methods (e.g. Ishizawa M., Kobayashi Y., Miyamura T., Matsuma, S: Simple procedure of DNA isolation from human serum. Nucl. Acids Res. 1991; 19:5792). The amplification is set up as follows:

cDNA Synthesis/1st PCR

Ten μl of the extracted RNA are mixed with 25 μl of 2× Reaction Mix (Life Technologies), 2 μl of primer 6 (50 pmol/μl; see SEQ ID No. 6 in the sequence listing), 2 μl of primer 7 (50 pmol/μl; see SEQ ID No. 7 in the sequence listing), 10 μl of water and 1 μl of SuperscriptII/Taq polymerase mix (Life Technologies) and the mixture is subjected to the following thermocycles;

1. Incubation for 1 hour at 50° C. in order to synthesize the cDNA

2. Initial denaturation for 2 minutes at 94° C.

3. 35 cycles of in each case 28 seconds at 94° C. (denaturation), 28 seconds at 50° C. (annealing) and 38 seconds at 60° C. (extension)

2nd PCR

Five μl of the 1st PCR mixture are mixed with 45 μl of a 2nd PCR reaction mixture [16.55 μl of water, 5 μl of 10× PCR buffer (Perkin Elmer), 3 μl of magnesium chloride (25 mM), 4 μl of dNTPs (2.5 mM), 8 μl of primer 8 (10 pmol/μ1; see SEQ ID No. 8 in the sequence listing), 8 μl of primer 4 (10 pmol/μl; see SEQ ID No. 4 in the sequence listing), 0.25 μl of the molecular beacon probe 9 (5 pmol/μl; see SEQ ID No. 9 in the sequence listing), 0.25 μl of Taq DNA polymerase (2.5 units, Perkin Elmer)] and the mixture is subjected to the following thermocycles:

1. Initial denaturation for 1 minute at 90° C.

2. 35 cycles of in each case 28 seconds at 94° C. (denaturation), 28 seconds at 56° C. (annealing) and 38 seconds at 72° C. (extension)

3. Cooling down to 20° C. within 10 minutes

Evaluation

The PCR reaction is evaluated in a fluorescence spectrometer. For this, the fluorescence is measured at the reporter wavelength (518 nm for FAM). A threshold value is calculated on the basis of the fluorescence of negative controls which do not contain any target sequence and unknowns are evaluated against this value.

Results

The results achieved in experiments carried out under the above-described conditions were equivalent, both as regards the detectability of HCV genotypes (isolates of genotypes 1 to 5 were tested) and as regards analytical sensitivity, to the results obtained by means of a nested PCR using autologous primers and probes.

The nucleic acid amplification method according to the invention does not require both primers in the primer pair employed to be heterologous. An amplification which is suitable for detecting and/or isolating the target nucleic acid can also be performed using a combination of an autologous primer and a heterologous primer.

The invention also relates to a reagent set for performing a PCR in which set one or both primers is/are heterologous. The invention additionally relates to a reagent set which, in addition to a abovementioned primers also contains an oligonucleotide probe which is derived from a genome of an organism of another species and is consequently heterologous and preferably present as a molecular beacon probe.

In the case of the oligonucleotides according to the invention, the universal base inosine can be used to compensate for any mismatches which prevent complete hybridization with the nucleotide sequence of the target nucleic acid. Incomplete hybridization with the target nucleic acid can also occur due to the presence of variants of the target nucleic acid which are as yet unknown. These variants can still be specifically detected by the heterologous primers and probes according to the invention because the universal base inosine is inserted into the primer or probe at the mismatch site. The process according to the invention consequently has a larger detection range than a process which operates exclusively with autologous primers and probes.

Patent claims for USA:

1. A process for finding heterologous oligonucleotide sequences for a nucleic acid amplification method, wherein
a) mutually overlapping oligonucleotide sequences are generated by fragmenting conserved regions of the target nucleic acid to be amplified,
b) these sequence fragments are used for finding similar DNA segments in Genbank or other DNA databases and suitably heterologous oligonucleotide sequences which are derived from organisms of other species are thereby identified, and
c) the heterologous oligonucleotide sequences which have been found are employed as primers and/or probes for isolating the target nucleic acid using a nucleic acid amplification method.

2. The process as claimed in claim 1, wherein mutually overlapping oligonucleotide sequences, which comprise from 30 to 50 bases, are generated by fragmenting conserved regions in a genome of a virus and heterologous oligonucleotide sequences, which are suitable for detecting the virus, are identified in a gene library.

3. The process as claimed in claim 1, wherein the mismatches which are present in the hybridizing, heterologous oligonucleotide sequences which have been found are replaced with a universal base (e.g. inosine) and complete hybridization with the nucleotide sequence of the target nucleic acid is thereby achieved.

4. A method for nucleic acid amplification, wherein the heterologous oligonucleotide sequences which have been obtained as claimed in claim 1 are employed as primers and/or probes for selectively isolating a predetermined target nucleic acid.

5. The method as claimed in claim 4, wherein a nucleic acid amplification method, such as the polymerase chain reaction (PCR), NASBA (=nucleic acid sequence-based amplification), TMA (transcription-mediated amplification) or LCR (ligase chain reaction), is employed for amplifying the target nucleic acid.

6. A reagent set for implementing a polymerase chain reaction, which comprises a pair of oligonucleotide primers which possess the sought-after DNA sequence and which have been derived from a genome present in an organism of another species.

7. The reagent set as claimed in claim 6, which additionally comprises an oligonucleotide probe which contains a heterologous DNA sequence which is derived from a genome of an organism of another species and which hybridizes with the target nucleic acid DNA sequence which is flanked by the primers.

8. The reagent set as claimed in claim 7, wherein the probe carries two fluorescent dyes (reporter and quencher) in the 5′ and 3′ positions, which dyes influence each other's fluorescence.

9. The reagent set as claimed in claim 6, wherein use is made of a primer which is labeled with two fluorescent dyes (reporter and quencher) and which does not hybridize completely with the DNA sequence to be amplified at the 3′ end.
